(19) United States
(12) Patent Application Publication
(10) Pub. No.: US 2006/0251724 A1
(43) Pub. Date: Nov. 9, 2006

(54) Method for preparing thermoformed compositions containing acrylic polymer binders, pharmaceutical dosage forms and methods of preparing the same

(76) Inventors: Thomas P Farrell, Warrington, PA (US); Kurt Fegely, Limerick, PA (US); Christopher R. Young, Austin, TX (US); Michael Crowley, Austin, TX (US); James W. McGinity, Austin, TX (US)

Correspondence Address: LUCAS & MERCANTI, LLP, 475 PARK AVENUE SOUTH, 15TH FLOOR, NEW YORK, NY, 10016, US

(21) Appl. No.: 10/554,677

(22) PCT Filed: May 6, 2004
(86) PCT No.: PCT/US04/14109

§ 371 Date: Oct. 26, 2005

Related U.S. Application Data
(60) Provisional application No. 60/468,625, filed on May 6, 2003.

(51) Int. Cl. A61K 9/14; C08L 33/04

(52) U.S. Cl. 424/487; 525/222

(57) ABSTRACT

Thermoformed or hot melt extruded pharmaceutical compositions containing an active pharmaceutical ingredient, acrylic enteric polymer and plasticizer are disclosed. Methods of making the same as well as various pharmaceutical dosage forms are also disclosed. In preferred aspects, a thermoformable mixture is in a powder form and includes an active ingredient, plasticizer, acrylic polymer and optional excipients which enhance the performance of the extrudate after being incorporated into a dosage form.

### CROSS-REFERENCE TO RELATED APPLICATIONS

This application claims the benefit of priority from U.S. Provisional Application Ser. No. 60/468,625, filed May 6, 2003, the disclosure of which is incorporated herein by reference.

### BACKGROUND OF THE INVENTION

1. Field of the Invention

The present invention relates to extrudable compositions useful in the pharmaceutical industry. The invention also relates to methods of preparing extruded excipients as well as various extrudates containing a pharmaceutically active ingredient.

2. Description of the Prior Art

Hot-melt extrusion (HME) is a widely applied processing technique used in the plastics industry to produce tubes, pipes, wires and films. For pharmaceutical systems, this method has been used to prepare granules, sustained-release tablets and transdermal drug delivery systems. Today, interest in hot-melt extrusion techniques is growing rapidly with over 100 papers published. The number of hot-melt extrusion patents issued for pharmaceutical systems has steadily increased since the early 1980's.

Hot-melt extrusion offers many advantages over traditional pharmaceutical processing techniques. Solvents and water are not necessary, reducing the number of processing steps and eliminating time-consuming drying steps. The active ingredients do not need to be compressible and the entire procedure is continuous and efficient. The intense mixing and agitation imposed by the rotating screw cause de-aggregation of suspended particles in the molten polymer resulting in a more uniform dispersion. Hot-melt extrusion also has been used to improve the bioavailability of drug substances by formation of molecular dispersions. All components must be thermally stable at the processing temperature during the short duration of the heating process. Thus, hot-melt extrusion requires a pharmaceutical grade polymer that can be processed at relatively low temperatures due to the thermal sensitivity of many drugs.

Many hot-melt extrusion processes have generally required elevated processing temperatures. These high temperatures, however, have been recognized by those in the pharmaceutical formulation arts to cause decomposition of the therapeutic agent and/or polymer carrier or carrier matrix.

One of the earlier attempts to employ an extrusion process in the preparation of a pharmaceutical formulation is found in U.S. Pat. No. 5,073,379. This patent describes a continuous process of extruding a polymer melt containing the active compound and forming the still plastic extrudate between a belt and a roller or two belts.

U.S. Pat. No. 6,051,253 discloses solid drug forms being produced by mixing and melting a pharmacologically acceptable polymeric binder and a pharmaceutical active ingredient, with or without conventional pharmaceutical additives, in the absence of a solvent to give a plastic mixture. The extrudate is shaped in two steps. The extrudate is broken into shaped articles and the shaped articles are rounded off in a second step in the plastic state.

One recent example of the efforts in this regard is found in PCT publication WO 02/35991 which discloses active agent containing spherical pellets being formed using a hot-melt extrusion process. Specifically, a thermoformable composition is obtained by extruding an active ingredient, a plasticizer, a polymer and optional excipients into ribbon-like extrudates which are then cut into pellets which undergo a spheronization process. Preferred polymers include various EUDRAGIT® brand products and the like which are selected of the basis of having a glass transition temperature (Tg) below the decomposition temperature of the active agent.

U.S. Pat. No. 6,488,963 to McGinity, et al. discloses pharmaceutical formulations containing a thermoformable mixture of a therapeutic compound and a high molecular weight poly(ethylene oxide) such as PEG in an essentially non-film like preparation and methods of preparing the same. The '963 patent, however, does not disclose preparing thermoformable pharmaceutical formulations containing enteric-like polymers as a part thereof.

Commonly assigned U.S. Pat. No. 6,420,473, the disclosure of which is incorporated herein by reference, describes fully-formulated, non-toxic, edible, enteric, film-coating, dry powder compositions based on an acrylic resins which are used to make aqueous enteric coating suspensions for coating pharmaceuticals. The coatings are insoluble in gastric juices of the stomach but intestinally soluble. Since the powder compositions, which are marketed by Colorcon of West Point, Pa. under the trademark ACRYL EZE®, were designed to be included in aqueous film coating systems, the compositions also included an alkalizing agent capable of reacting with the acrylic resin and a detackifier. There was no disclosure or suggestion that these film coating systems could be used in hot melt extrusion or other thermoforming processes.

In spite of the foregoing, there is still a need in the art to develop additional controlled-release pharmaceutical formulations. The present invention addresses this need.

### SUMMARY OF THE INVENTION

In one aspect of the invention there is provided a thermoformable composition suitable for use in pharmaceutical formulations. The thermoformable or extrudable composition is preferably powder-based and includes i) a thermoformable or extrudable acrylic polymer binder and ii) an effective amount of an acrylic polymer plasticizer. The acrylic polymer binder includes an acrylic resin having:

a) from 20 to 85 percent by weight of at least one alkyl acrylate or alkyl methacrylate moiety,

b) from 80 to 15 percent by weight of at least one vinyl or vinylidene moiety having a carboxylic acid group capable of salt formation, and

c) from 0 to 30 percent by weight of at least one other vinyl or vinylidene moiety copolymerizable with a) and b).

The composition is ready for hot-melt extrusion or other thermoforming processes described herein, preferably after being mixed with a pharmaceutically active composition and one or more optional pharmaceutical excipients.

In further aspects of the invention, there are provided methods of making the powder-based thermoformable or extrudable composition, methods of making thermoformed compositions, methods of making pharmaceutical dosage forms containing the extruded or thermoformed compositions as well as the resultant pharmaceutical dosage forms, i.e. tablets, capsules, etc.

As a result of the present invention, several advantages are provided. First, it has been surprisingly found that polymeric coating compositions which were thought to be only useful for the enteric coating of tablets and the like can now be employed in hot melt extrusions processes without loss of their extended release properties. In addition, it has also been unexpectedly found that extruded compositions which contain the inventive mixture of the enteric polymeric coatings and plasticizers can provide the artisan with zero order or near zero order in vitro release profiles.

Furthermore, in some preferred embodiments of the invention, such as those in which the thermoformable powder includes a pre-plasticized acrylic polymer and detackifier, it has been surprisingly found that additional amounts of plasticizer can be added and rapidly assimilated for efficient throughput. In addition, regardless of the embodiment, it has been found that the compositions of the present invention provide extruded products, which not only have a high degree of content uniformity, but also can be made in a single pass through the extruder.

### BRIEF DESCRIPTION OF THE DRAWINGS

FIG. 1 is a graph illustrating the in vitro release profile of guaifenisin from tablets containing the hot melt extruded composition of Examples 5-6.

FIG. 2 is a graph illustrating the in vitro release profiles of theophylline from tablets containing hot melt extruded compositions of Examples 7-8.

FIG. 3 is a graph illustrating the in vitro release profiles of theophylline from tablets containing the hot melt extrusion compositions of Examples 9-10.

FIG. 4 is a graph showing the time release profile of tablets containing various amounts of a carbomer and prepared according to Examples 11-13.

FIG. 5 is a graph illustrating the in vitro release profiles of theophylline from tablets containing the hot melt extrusion compositions of Examples 15-17.

FIG. 6 is a graph illustrating the in vitro release profiles of theophylline from tablets containing the hot melt extrusion compositions of Examples 18-20.

FIG. 7 is a graph illustrating the in vitro release profiles of theophylline from tablets containing the hot melt extrusion compositions comprising Acryl-EZE and varying amounts of Carbopol 974P, before and after storage for 3 months at 40° C./75% RH.

### DETAILED DESCRIPTION OF THE INVENTION

A. Hot-Melt Extrusion Process

As used herein, the term “thermoformable” refers to a compound or formulation that may be thermoformed or capable of being processed by melting or rendered flowable during thermal processing, i.e. extruded under a combination of increased temperature and/or pressure. A thermoformable polymer is one that is sufficiently rigid at standard ambient temperature and pressure but is capable of deformation or forming a semi-liquid state under elevated heat or pressure, either alone or, as is preferably the case herein, with a plasticizer of the type described below.

Although the inventive process is called a thermoforming or hot-melt extrusion process, other equivalents processes know to those of ordinary skill, such as injection molding, hot dipping, melt casting, melt granulation and compression molding may be used. By using any of these methods, the formulation may be shaped as needed according to the desired mode of administration, e.g. tablets, pills, lozenges, suppositories and the like.

The hot-melt extrusion process employed in some embodiments of the invention is conducted at an elevated temperature, i.e. the heating zone(s) of the extruder is/are above room temperature (about 20° C.). It is important to select an operating temperature range for the extrusion process that will minimize the degradation or decomposition of the therapeutic compound/pharmaceutical active during processing. Such temperatures will, of course, vary depending upon the active ingredient and will be apparent to those of ordinary skill. While no specific temperature range is required, it is contemplated that in most aspects of the invention, the operating temperature range will be generally in the range of from about 60° C. to about 160° C. as determined by the setting for the extruder heating zone(s).

In some embodiments of the invention, the hot-melt extrusion may be conducted employing a slurry, solid, suspension, liquid, powdered or other such feed comprising the acrylic polymer binder, plasticizer, pharmaceutically active ingredient, if included, and all optionally present excipients. In most aspects, dry feed is preferably employed in the processes of the present invention.

The hot-melt extrusion process is generally described as follows:

The desired amount of an enteric acrylic polymer is mixed with the plasticizer, pharmaceutically active composition or active pharmaceutical ingredient (hereinafter API) and any other optionally included excipients before being introduced into the extruder. In some embodiments, the therapeutic compound functions unexpectedly as a non-traditional plasticizer, eliminating the need for a separate (additional) plasticizer in compositions of the present invention. Guaifenesin is one such API and others having solubility parameters close to those of the acrylic polymers or having the ability to lower the Tg or softening point of the polymers are contemplated. In many alternative aspects, however, the API is combined with the other ingredients to form a mixture, which is usually a dry powder. It is to be noted, however, when the plasticizer is a liquid such as triethyl citrate (TEC), it is preferably added geometrically to the dry ingredient components before being extruded. The mixture is then placed in the extruder hopper and passed through the heated area (zone(s)) of the extruder at a temperature which will melt or soften the acrylic enteric polymer and plasticizer to form a matrix throughout which the API is dispersed. The molten or softened mixture then exits via a die, or other such element, at which time, the transformed mixture (now called the extrudate) begins to harden. Since the extrudate is still warm or hot upon exiting the die, it may be easily shaped, molded, chopped, ground, molded, spheronized into beads, cut into strands, tableted or otherwise processed to the desired physical form, i.e. pharmaceutical dosage form.

Any art recognized extruder may be used to practice the invention. Suitable and non-limiting examples of such devices include those commercially available and equipped to handle dry feed, having a solid conveying zone, one or multiple heating zones, and an extrusion die. One such device is a Microtruder® RCP-0750 single-screw extruder available from Randcastle of Cedar Grove, N.J. Another device useful for carrying out the present invention is a two stage single screw extruder manufactured by C. W. Brabender Instruments Inc. of New Jersey. Twin or multiple screw extruders may also be employed, depending upon the needs of the artisan. It is particularly advantageous for the extruder to possess multiple separate temperature controllable heating zones.

Many conditions may be varied during the extrusion process to arrive at a particularly advantageous formulation. Such conditions include, by way of example, formulation composition, feed rate, operating temperature, extruder screw RPM, residence time, die configuration, heating zone length and extruder torque and/or pressure. Methods for the optimization of such conditions are known to the skilled artisan.

B. Acrylic Polymer

In preferred aspects of the invention, the extrudable acrylic polymer binder portion contains an acrylic resin, containing:

a) from 20 to 85 percent by weight of at least one alkyl acrylate or alkyl methacrylate moiety;

b) from 80 to 15 percent by weight of at least one vinyl or vinylidene moiety having a carboxylic acid group capable of salt formation; and

c) from 0 to 30 percent by weight of at least one other vinyl or vinylidene moiety copolymerizable with a) and b).

Such formulations are described, for example, in commonly assigned U.S. Pat. No. 6,420,473, the contents of which are incorporated herein by reference. Such acrylic enteric polymers are also available from Colorcon as ACRYL-EZE® and may include auxiliary ingredients such as an alkalizing agent and a detackifier as well as many other optional ingredients described below. The alkalizing agent is capable of reacting with the acrylic resin portion of the acrylic polymer such that, after reaction, 0.1 to 10 mole percent of the acidic groups in the vinyl or vinylidene moiety having a carboxylic acid group capable of salt formation are present in the salt form. It is to be understood that the invention is in no way limited to the presently commercially available Acryl-EZE formulations and that many extrudable formulations are contemplated in which the desired enteric acrylic polymer binder, plasticizer and all desired optional ingredients are individually selected and pre-mixed prior to extrusion. The commercially available Acryl EZE formulations, however, provide the artisan with readily available extrudable acrylic enteric polymer formulations for use in carrying out the present invention.

In accordance with the invention, the thermoformable acrylic polymer binder is preferably a dry powder composition which comprises an acrylic resin. An acrylic resin meeting the requirements set forth above is available from Rohm Pharma GmbH (Germany) under the tradename EUDRAGIT L100-55 and is based upon copolymers of ethylacrylate and methacrylic acid. See also U.S. Pat. No. 4,520,172, the disclosure of which is incorporated herein by reference.

The extrudable acrylic polymer binder is an acrylic resin, which comprises at least one vinyl or vinylidene moiety having a carboxylic acid group capable of salt formation. The acrylic resin may comprise of at least one vinyl or vinylidene moiety having a carboxylic acid group capable of salt formation and at least one alkyl acrylate or alkyl methacrylate moiety. The acrylic resin also may comprise of at least one vinyl or vinylidene moiety having a carboxylic acid group capable of salt formation, at least one alkyl acrylate or alkyl methacrylate moiety, and at least one other vinyl or vinylidene moiety copolymerizable with a) the alkyl acrylate or alkyl methacrylate moiety and b) the vinyl or vinylidene moiety having a carboxylic acid group capable of salt formation. Further, the acrylic resin may comprise of at least one vinyl or vinylidene moiety having a carboxylic acid group capable of salt formation and at least one other vinyl or vinylidene moiety copolymerizable with the vinyl or vinylidene moiety having a carboxylic acid group capable of salt formation.

Preferably, the acrylic polymer binder is an acrylic resin which is comprised of: (1) from 20 to 85 percent by weight of at least one alkyl acrylate or alkyl methacrylate moiety; (2) from 80 to 15 percent by weight of at least one vinyl or vinylidene moiety having a carboxylic acid group capable of salt formation; and (3) from 0 to 30 percent by weight of at least one other vinyl or vinylidene moiety copolymerizable with (1) and (2). In a particularly preferred embodiment of this invention, the alkyl acrylate (1) is ethyl acrylate, and the vinyl moiety (2) is methacrylic acid. EUDRAGIT L100-55 powder is one example of a copolymer system meeting this definition.

Preferably, the acrylic resin comprises from about 10% to about 80% by weight, preferably from about 15% to about 70% by weight, and most preferably about from about 20% to about 60% by weight of the extrudable composition of the invention.

The optional alkalizing agent mentioned above may be a bicarbonate, a carbonate, a phosphate, or a hydroxide of sodium or potassium, magnesium carbonate, magnesium hydroxide, ammonium carbonate, ammonium bicarbonate, magnesium oxide, calcium hydroxide, or mixtures thereof. The quantity of alkalizing agent used is directly dependent on the amount of carboxylic acid-bearing vinyl or vinylidene moiety present in the acrylic resin. Specifically, said alkalizing agent is added in a quantity such that, after reaction with the acrylic resin, 0.1 to 10 mole percent of the acidic groups are present in the salt form.

The detackifier mentioned above may be talc, aluminum hydrate, glyceryl monostearate, kaolin, or mixtures thereof. Preferably, the detackifier comprises about 5% to about 40% by weight of the extrudable composition of the invention.

In still further aspects of the invention, the acrylic polymer binder can be included as part of a ready to use pre-blend which can be combined with any API and other optional ingredients. The pre-blends (alone) therefore contain: from about 20 to about 80% by weight Eudragit L100-55; from about 15 to about 60% by weight triethyl citrate; and from about 19 to about 76% by weight talc.

C. Plasticizers

As used herein, the term “plasticizer” includes all compounds capable of plasticizing the acrylic polymer binders described above. The plasticizer should be able to lower the glass transition temperature or softening point of the acrylic polymer in order to allow for lower processing temperature, extruder torque and pressure during the hot-melt extrusion process.

A non-limiting list of suitable plasticizers include triethylcitrate, glyceryl monostearate, glyceryltriacetate, acetyltriethylcitrate, dibutyl sebacate, diethylphthalate, polyethylene glycols, glycerol, castor oil, or mixtures thereof. Preferably, the plasticizer is triethylcitrate. It is also contemplated and within the scope of the invention, that a combination of plasticizers may be used in the present formulation. Other plasticizers useful in the invention include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene oxides) (average molecular weight less than about 500,000), ethylene glycol, propylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, triethyl citrate, acetyl triethyl citrate, tributyl citrate and allyl glycolate. All such plasticizers are commercially available from sources such as Aldrich or Sigma Chemical Co.

The amount of plasticizer included in the thermoformable compositions of the invention can range from about 4.0 to about 40% by weight. Preferably, the amount is from about 7 to about 35% by weight while most preferably, the amount is from about 10 to about 30% by weight. The amount of plasticizer used in the formulation will also depend upon its composition, physical properties, effect upon the acrylic polymer, interaction with other components of the formulation, and other factors to be considered in the preparation of pharmaceutical formulations.

D. Therapeutic Preparations

As mentioned above, the API included in the compositions of the present invention can vary widely according to the needs of the artisan. The only limitation thereon is that the API must be capable of undergoing the extrusion process described herein without undergoing significant decomposition/degradation. The amount of API included in the extrudable and extruded compositions of the present invention will generally be amounts ranging from about 0.001 to about 85% by wt., depending on the desired release profile, the pharmacological activity and toxicity of the therapeutic compound and other such considerations. Preferably, however, the amount ranges from 1.0 to about 60 and most preferably from about 3.0 to about 10% by wt.

As used herein, the term “therapeutic compound” or “API” is taken to mean an organic chemical substance having desired beneficial and therapeutic effects in mammals. Such compounds are generally classified as pharmaceuticals or biologicals. As long as the therapeutic compound can diffuse from the formulation when exposed to a biological fluid, its structure is not especially critical. The therapeutic compounds contemplated within the scope of the invention include hydrophobic, hydrophilic and amphiphilic compounds. They may be in their free acid, free base, or pharmaceutically acceptable salt forms. They may be derivatives or prodrugs of a given pharmaceutical. It will be appreciated that certain therapeutic compounds used in the present invention may contain an asymmetrically substituted carbon atom, and may be isolated in optically active or racemic forms. It is well known in the art how to prepare optically active forms, such as by resolution of racemic forms or by synthesis, from optically active starting materials. Also, it is realized that cis and trans geometric isomers of the therapeutic compounds are described and may be isolated as a mixture of isomers or as separated isomeric forms. All chiral, diastereomeric, racemic forms and all geometric isomeric forms of a structure are intended, unless the specific stereochemistry or isomer form is specifically indicated.

It is not necessary for the therapeutic compound to be soluble in any given formulation component. The therapeutic compound may be either dissolved, partially dissolved or suspended in the polymer matrix of the formulation. It is necessary for the therapeutic compound to be stable during the hot-melt extrusion process conditions used. By stable, it is meant that a significant portion of the therapeutic compound will not be significantly degraded or decomposed throughout the hot-melt extrusion process.

The therapeutic compounds which may be thermoformed in the formulation of the invention may be used for treating indications such as, by way of example and without limitation, inflammation, gout, hypercholesterolemia, microbial infection, AIDS, tuberculosis, fungal infection, amoebic infection, parasitic infection, cancer, tumor, organ rejection, diabetes, heart failure, arthritis, asthma, pain, congestion, urinary tract infections, vaginal infection, seizure related disorder, depression, psychosis, convulsion, diabetes, blood coagulation, hypertension and birth control.

The following therapeutic compounds are examples of the API's which can be administered by the pharmaceutical formulation of the present invention. This list is illustrative and not exclusive:

(1) analgesics such as aspirin, acetaminophen, deflunisal and the like;

(2) anesthetics such as lidocaine, procaine, benzocaine, xylocaine and the like;

(3) antiarthritics and anti-inflammatory agents such as phenylbutazone, indomethacin, sulindac, dexamethasone, ibuprofen, allopurinol, oxyphenbutazone probenecid, cortisone, hydrocortisone, betamethasone, dexamethasone, fluocortolone, prednisolone, triamncinolone, indomethacin, sulindac and its salts and corresponding sulfide and the like;

(4) antiasthma drugs such as theophylline, ephedrine, beclomethasone dipropionate, epinephrine and the like;

(5) urinary tract disinfectives such as sulfamethoxazole, trimethoprim, nitrofurantoin, norfloxicin and the like;

(6) anticoagulants such as heparin, bishydroxy coumarin, warfarin and the like;

(7) anticonvulsants such as diphenylhydantoin, diazepam and the like;

(8) antidepressants such as amitriptyline, chlordiazepoxide, perphenazine, protriptyline, imipramine, doxepin and the like;

(9) agents useful in the treatment of diabetics and regulation of blood sugar, such as insulin, tolbutamide tolazamide, somatotropin, acetohexamide, chlorpropamide and the like;

(10) antineoplastics such as adriamycin, fluouracil, methotrexate, asparaginase and the like;

(11) antipsychotics such as prochlorperazine, lithium carbonate, lithium citrate, thioridazine, molindone, fluphenazine, trifluoperazine, perphenazine, amitriptyline, triflupromazine and the like;

(12) antihypertensives such as spironolactone, methyldopa, hydralazine, clonidine, chlorothiazide, deserpidine, timolol, propranolol, metaprotol, prazosin hydrochloride, reserpine and the like;

(13) muscle relaxants such as mephalan, danbrolene, cyclobenzaprine, methocarbarnol, diazepam, succinoyl chloride and the like;

(14) antiprotozoals such as chloramphenicol, chloroquine, trimethoprim and sulfamethoxazole;

(15) spermicidals such as nonoxynol;

(16) antibacterial substances such as beta-lactam antibiotics, tetracyclines, chloramphenicol, neomycin, cefoxitin, thienamycin, gramicidin, bacitracin, sulfonamides, aminoglycoside antibiotics, tobramycin, nitrofurazone, nalidixic acid and analogs and the antimicrobial combination of fludalanine/pentizidone;

(17) antihistamines and decongestants such as perilamine, chlorpheniramine, tetrahydrozoline and antazoline;

(18) antiparasitic compounds such as ivermectin; and

(19) antiviral compounds such as acyclovir and interferon.

E Optional Ingredients

The extrudable compositions of the present invention as well as the extruded products and pharmaceutical dosage forms may also include one or more functional excipients. These excipients are broadly classified as release-modifying agents, bulking agents, processing agents and miscellaneous additives. The selection and use of various excipients can impart specific properties to the compositions of the present invention in a manner similar to those in traditional dosage forms. A non-limiting list of such excipients include release rate modifiers, pigments, flow aids, surfactants, anti-agglomerating agents, secondary binders, secondary detackifiers, etc. and the like.

The release rate modifier is a substance, which when added to the extrudable ingredients prior to extrusion, has an effect on the release of the API from the extruded matrix. For most aspects of the invention, the release rate modifier is a substance which prolongs the rate of release of the API from the extruded polymer matrix. The following are a non-limiting list of the substances suitable for this purpose: hydroxypropylcellulose (HPC), poly(ethylene oxide) (PEO), hydroxypropyl methylcellulose (HPMC) or hypromellose (Methocel®), ethylcellulose, cellulosic polymers, acrylic polymers, fat, waxes, lipids, polycarbophils, carbomers, polysaccharides and mixtures thereof. In preferred aspects, the release rate modifier is a carbomer such as Carbopol 934, a product of Noveon, Cleveland, Ohio. Other Carbopols including 940, 941, 974, 980 and 981 may also be used.

The amount of release rate modifier included in the extrudable compositions of the present invention ranges from about 1 to about 40% by weight, and is preferably from about 2 to about 30% by weight.

The pigment may be an FD&C or a D&C lake, titanium dioxide, magnesium carbonate, talc, pyrogenic silica, iron oxides, channel black, riboflavin, carmine 40, curcumin, annatto, insoluble dyes, pearlescent pigments based on mica and/or titanium dioxide or mixtures thereof. Other examples of suitable pigments are listed in Jeffries U.S. Pat. No. 3,149,040; Butler, et. al. U.S. Pat. No. 3,297,535; and Colorcon U.S. Pat. No. 3,981,984; all of which are incorporated herein by reference. The pigment may also include lake blends which contain a plasticizer and OPADRY pigmented coating compositions, some of which are disclosed in U.S. Pat. No. 4,543,370, which is incorporated herein by reference. Preferably, the pigment comprises 0% to about 50% by weight of the extrudable composition.

The flow aid may be silica such as fumed silica, supplied under the tradename Cab-O-Sil by Cabot, Inc. The flow aid imparts flowability to the powdered composition during dry blending and subsequent transferring from the blender to a storage container. Preferably, the flow aid comprises 0% to about 3% by weight of the extrudable composition.

The surfactant may be sodium lauryl sulfate, dioctyl sodium sulfosuccinate, polysorbates such as Tween 80, polyols such as sorbitol and the like or mixtures thereof. Other wetting agents such as glycerine. PEG, PPG, etc. are contemplated. Preferably, the surfactant and/or wetting agent comprise from 0% to about 5% by weight of the extrudable composition.

The anti-agglomerating agent may be kaolin. The quantity of anti-agglomerating agent in the inventive dry coating composition ranges from 0% to about 40% by weight of the extrudable composition. Beneficially, kaolin serves both as an anti-agglomerating agent and a detackifier.

The secondary binder may be xanthan gum, sodium alginate, pre-gelatinized starch, propylene glycol alginate, hydroxypropylmethylcellulose (HPMC), hydroxyethylecellulose (HEC), sodium carboxymethylcellulose (sodium CMC), polyvinylpyrrolidone (PVP), Konjac flour, carrageenan, other film-forming polymer or mixtures thereof. Preferably, the amount of secondary film former in the coating composition ranges from 0% to about 5% by weight of the dry coating composition of the invention.

The second detackifier may be sodium sulfate, calcium sulfate, calcium chloride, other inorganic or organic water-sequestering agents or mixtures thereof. Preferably, the amount of secondary detackifier in the coating composition ranges from 0% to about 5% by weight of the inventive dry coating composition of the invention.

### EXAMPLES

The following examples serve to provide further appreciation of the invention but are not meant in any way to restrict the effective scope of the invention. The following materials were employed:

| | | | LOT |
|---|---|---|---|
| MATERIAL | FUNCTION | SUPPLIER | NUMBER |
| Acryl-EZE | Thermal Binder | Colorcon | WP547929 |
| formula | | | |
| 93O18359 | | | |
| Theophylline | Active | Spectrum | RR0189 |
| Guaifenesin | Active | Spectrum | RE1530 |
| Triethyl | Plasticizer | Morflex | 37069 |
| Citrate | | | |
| Polyethylene | Plasticizer | Dow | B684 |
| Glycol 8000 | | | |
| Glycerol | Lubricant/Excipient | Condea | 909308 |
| Monostearate | | | |
| Carbomer 934 | Excipient | Spectrum | PJ0704 |
| Methocel | Excipient | Dow | QB22012N03 |
| K4M | | | |
| Carbopol | Excipient | Noveon | CC31NAB652 |
| 974P | | | |

### Hot-Melt Extrusion

Dry powder formulations containing Acryl-EZE® alone and powder blends containing Acryl-EZE, API and functional excipients were mixed for 5 minutes in a ceramic mortar and pestle prior to hot-melt extrusion. When the liquid plasticizer TEC was included in the formulation, the plasticizer was added geometrically to Acryl-EZE® in the mortar before mixing for 5 minutes. The dry powder formulations were extruded using a Randcastle Microtruder® RCP-0750 (Cedar Grove, N.J.) single-screw extruder. The extruder was equipped with a Nitralloy 135M screw (3:1 compression ratio with flight configuration containing feed, compression and mixing sections) and a cylindrical die (6 mm in diameter). The screw speed employed for all formulations was 15-20 rpm. Temperature of the extruder barrel zones and die were varied using external temperature controllers. The formulation was fed into the hopper after the extruder zones and die had equilibrated to the set temperatures. The extrudates were cooled to 25° C. and then manually cut into tablets. The guaifenisin tablets weighed approximately 280 mg, while the theophylline tablets weighed approximately 215 mg.

Drug Release Studies

Where carried out, the dissolution testing was performed according to Apparatus 2 (paddle method) of USP 24 on a Van Kel VK7000 Dissolution Tester equipped with an auto sampler (Model VK 8000). Enteric dissolution testing was performed according to Method B of USP 24, which included 2 hours in an acid stage (pH 1.2, 0.1 N HCl) followed by 8 hours in a buffer stage (pH 6.8, 50 mmol phosphate buffered solution). The dissolution vessel volume of 900 mL was maintained at 37° C. and agitated at 50 rpm. Samples were removed at specified time points over the 10 hour period.

Samples were analyzed for drug content using a Waters high performance liquid chromatography (HPLC) system with a photodiode array detector (Model 996) extracting at 276 nm for guaifenisin and 281 nm for theophylline. Samples were pre-filtered through a 0.2 μm membrane (Gelman Laboratory, GHP Acrodisc) to remove insoluble excipients. An auto sampler (Model 717plus) was used to inject 20 μL samples. The data were collected and integrated using Empower® Version 5.0 software. The column used for guaifenisin analysis was an Alltech Alltima™ Cl₈ 10 μm, 250×4.1 mm. The mobile phase contained a mixture of water:methanol:glacial acetic acid in volume ratios of 600:400:15. The solvents were vacuum filtered through a 0.45 μm nylon membrane and degassed using a Waters In-Line Degasser AF. The flow rate was 1.5 mL/min. The retention time of the guaifenisin was 3.5 minutes. Linearity was demonstrated from 2 to 200 mg/μL (R²≧0.997) and injection repeatability was 0.35% relative standard deviation for 10 injections. The column used for theophylline analysis was an Alltech Inertsil™ ODS-3 3 μm, 150×4.6 mm. The mobile phase contained a mixture of water:acetonitrile:glacial acetic acid in volume ratios of 845:150:5 and 1.156 g/L of sodium acetate trihydrate. The retention time of the theophylline was 3.6 minutes. Linearity was demonstrated from 1 to 100 mg/μL (R²≧0.998) and injection repeatability was 1% relative standard deviation for 6 injections.

### Examples 1-2

### Comparative

In these examples, the acrylic enteric polymer containing product Acryl-EZE was extruded alone.

### Example 1

| Component | Percent |
|---|---|
| Acryl-EZE | 100% |

Processing Parameters

| | | |
|---|---|---|
| Zone 1 | 110° | C. |
| Zone 2 | 125° | C. |
| Zone 3 | 130° | C. |
| Die | 135° | C. |
| Screw Speed | 15 | rpm |
| Drive Amps | 3-4 | Amp |
| Pressure | 500 | PSI |

Comments: poor hopper flowability, some die swelling, slow barrel flow, brittle

### Example 2

| Component | Percent |
|---|---|
| Acryl-EZE | 100% |

Processing Parameters

| | | |
|---|---|---|
| Zone 1 | 130° | C. |
| Zone 2 | 145° | C. |
| Zone 3 | 150° | C. |
| Die | 160° | C. |
| Screw Speed | 19 | rpm |
| Drive Amps | 0.34 | Amp |
| Pressure | 1000 | PSI |

Comments: poor hopper flowability, slow/no barrel flow

### Example 3

In this example, a plasticizer triethyl citrate (TEC) was added to the mixture prior to the extrusion.

| Component | Percent |
|---|---|
| Acryl-EZE | 80% |
| TEC | 20% |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 19 | rpm |
| Drive Amps | 0.35 | Amp |
| Pressure | 1000 | PSI |

Comments: excellent product, good flow, flexible, minimal die swelling

### Example 4

### Comparative

In this example, no plasticizer was used and theophylline was included as the API to the mixture prior to the extrusion.

| Component | Percent |
|---|---|
| Acryl-EZE | 80% |
| Theophylline | 20% |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 19 | rpm |
| Drive Amps | 0.4 | Amp |
| Pressure | 2000 | PSI |

Comments: product did not exit die and theophylline does not function as a plasticizer

### Examples 5-6

In these examples, no plasticizer was used and guaifenisin was included as the API to the mixture prior to the extrusion.

### Example 5

| Component | Percent |
|---|---|
| Acryl-EZE | 80% |
| Guaifenesin | 20% |

Processing Parameters

| | | |
|---|---|---|
| Zone 1 | 60° | C. |
| Zone 2 | 85° | C. |
| Zone 3 | 90° | C. |
| Die | 70° | C. |
| Screw Speed | 19 | rpm |
| Drive Amps | 0.22 | Amp |
| Pressure | 200 | PSI |

Comments: product very flexible, but not as soft and sticky with lower die temperature

### Example 6

| Component | Percent |
|---|---|
| Acryl-EZE | 80% |
| Guaifenesin | 20% |

Processing Parameters

| | | |
|---|---|---|
| Zone 1 | 60° | C. |
| Zone 2 | 80° | C. |
| Zone 3 | 80° | C. |
| Die | 70° | C. |
| Screw Speed | 19 | rpm |
| Drive Amps | 0.22 | Amp |
| Pressure | 200 | PSI |

Comments: product very flexible, but slight die swell

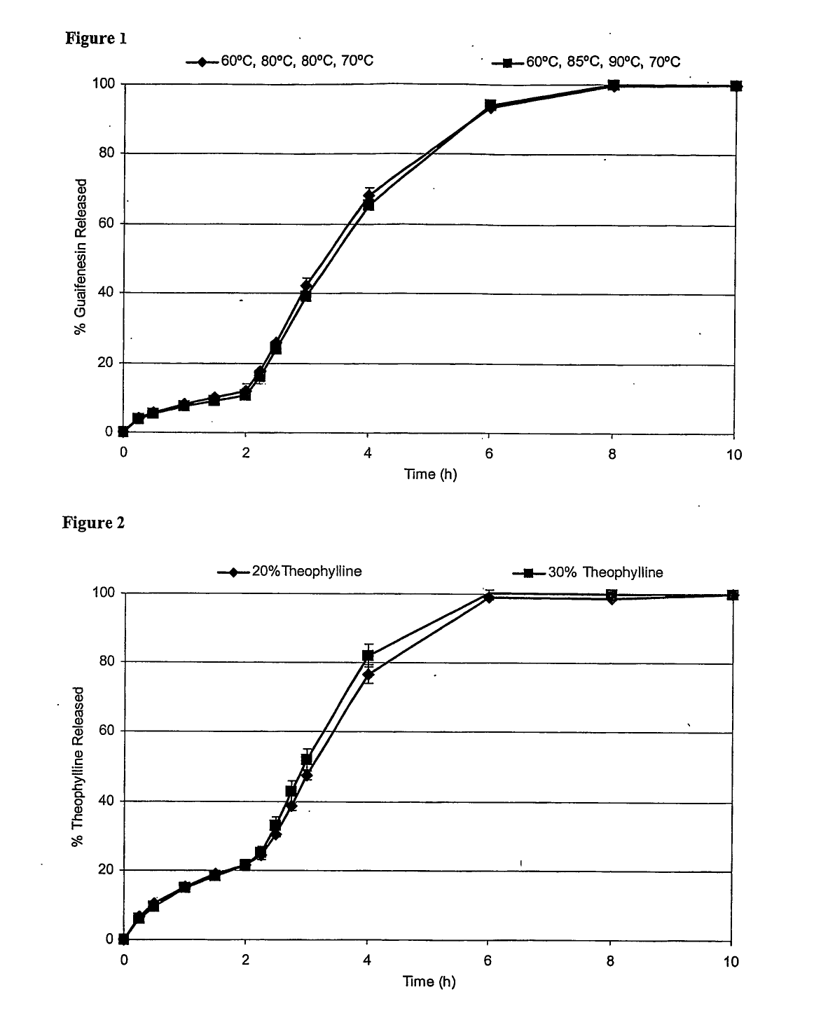

Turning now to FIG. 1, a review of the influence processing temperature has on the guaifenisin release profile of thermoformed tablets is provided. In each case, the tablets were prepared from an extrudate containing 80% Acryl-EZE and 20% guaifenisin. Dissolution conditions: 2 hours in pH 1.2 medium followed by 8 hours in pH 6.8 medium, 900 nm, 37° C., 50 rpm, n=6. It can be seen that the changes in the extruder zone temperature had a minor effect on the in vitro dissolution of the tablets in this example.

### Examples 7-8

In these examples, polymer, plasticizer and API were included.

### Example 7

| Component | Percent | |
|---|---|---|
| Acryl-EZE | 64% | |
| TEC | 16% | (25% based on |
| | | Acryl-EZE) |
| Theophylline | 20% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.2 | Amp |
| Pressure | 1500 | PSI |

Comments: good product, flexible, no die swelling

### Example 8

| Component | Percent | |
|---|---|---|
| Acryl-EZE | 56% | |
| TEC | 14% | (25% based on |
| | | Acryl-EZE) |
| Theophylline | 30% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 15 | rpm |
| Drive Amps | 0.6 | Amp |
| Pressure | 2000 | PSI |

Comments: flow reduced and less flexible than 20% theophylline

Turning now to FIG. 2, release profiles for tablets prepared to include the products of Examples 7 and 8 are provided. Specifically, evidence was sought to see if there was any difference observed when the drug: polymer ratio was changed from Example 7 to Example 8. Dissolution conditions: 2 hours in pH 1.2 medium followed by 8 hours in pH 6.8 medium, 900 mL, 37° C., 50 rpm, n=6. It can be seen that the amount of drug released between about 2 and 4 hours increased with the higher amount of drug included in the extrusion process.

### Examples 9-10

In these examples, polymer, plasticizer, API and glycerol monostearate (GMS) were included.

### Example 9

| Component | Percent | |
|---|---|---|
| Acryl-EZE | 56% | |
| TEC | 14% | (25% based on |
| | | Acryl-EZE) |
| Theophylline | 20% | |
| GMS | 10% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 15 | rpm |
| Drive Amps | 0.35 | Amp |
| Pressure | 1800 | PSI |

Comments: reduced flow rate, flexible product

### Example 10

| Component | Percent | |
|---|---|---|
| Acryl-EZE | 60% | |
| TEC | 15% | (25% based on |
| | | Acryl-EZE) |
| Theophylline | 20% | |
| GMS | 5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 18 | rpm |
| Drive Amps | 0.3 | Amp |
| Pressure | 1600 | PSI |

Comments: reduced flow rate but better than 10% GMS, flexible product

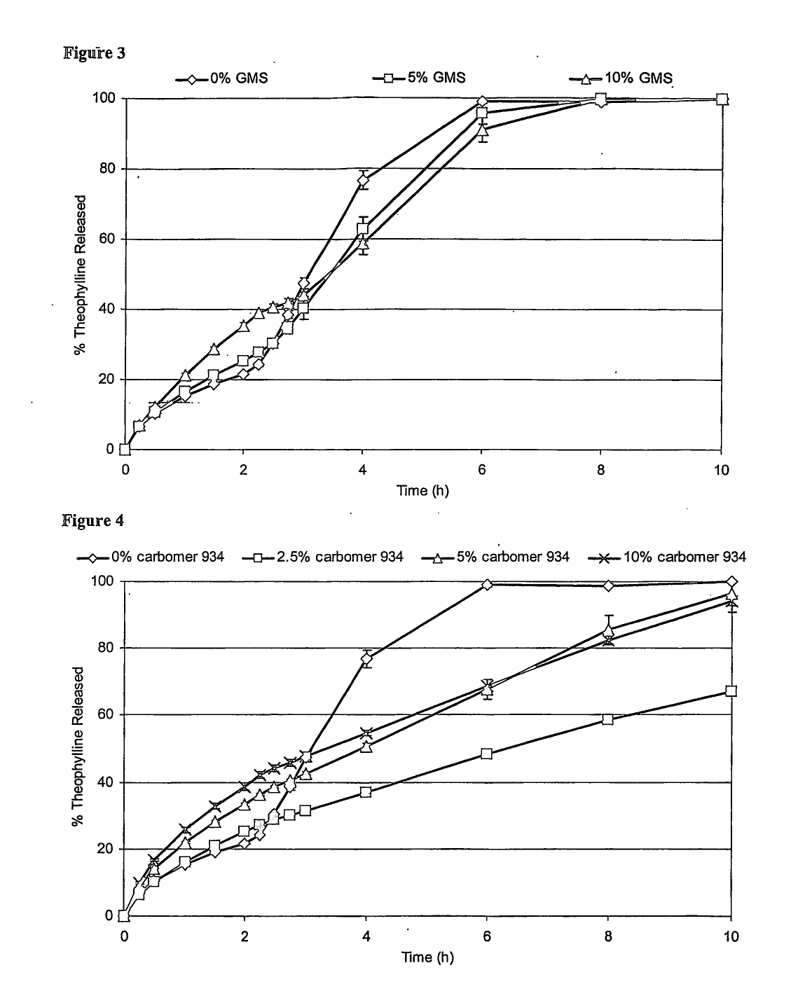

Turning now to FIG. 3, the effect of including GMS is illustrated. Using the product of example 7 as the product having 0% GMS as a baseline, the release profiles of the products of Examples 8 and 9 were compared. Dissolution conditions: 2 hours in pH 1.2 medium followed by 8 hours in pH 6.8 medium, 900 mL, 37° C., 50 rpm, n=6. It can be seen that the presence of the GMS reduces the amount of theophylline released, particularly after about 3 hours. Complete release of the drug is delayed slightly from 6 hours to about 8 hours. The amount of GMS included has some effect over this time period in reducing the release rate, but the doubling of the amount used (Ex. 8) did not extend the period over which the drug was released when compared to the amount used in Example 9.

### Examples 11-13

In these examples, polymer, plasticizer, API and a release rate modifier were included.

### Example 11

| Component | Percent | |
|---|---|---|
| Acryl-EZE | 56% | |
| TEC | 14% | (25% based on |
| | | Acryl-EZE) |
| Theophylline | 20% | |
| Carbomer 934 | 10% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 15 | rpm |
| Drive Amps | 0.25 | Amp |
| Pressure | 1500 | PSI |

Comments: some reduced flow, flexible product, rough surface

### Example 12

| Component | Percent | |
|---|---|---|
| Acryl-EZE | 60% | |
| TEC | 15% | (25% based on |
| | | Acryl-EZE) |
| Theophylline | 20% | |
| Carbomer 934 | 5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 18 | rpm |
| Drive Amps | 0.2 | Amp |
| Pressure | 1000 | PSI |

Comments: good flow, flexible product

### Example 13

| Component | Percent | |
|---|---|---|
| Acryl-EZE | 62% | |
| TEC | 15.5% | (25% based on |
| | | Acryl-EZE) |
| Theophylline | 20% | |
| Carbomer 934 | 2.5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.1 | Amp |
| Pressure | 1000 | PSI |

Comments: good flow, flexible product

Referring now to FIG. 4, the influence of carbomer 934 on the theophylline release profile of thermoformed tablets prepared as described above is illustrated. The baseline formulation was again the product of Example 7, e.g. 0% carbomer 934, 20% Theophylline, 64% Acryl-EZE 16% TEC (25% based on Acryl-EZE.

It can be seen that the presence of the carbomer 934 in the extrudate significantly extends the time over which the drug is released. The dissolution conditions: 2 hours in pH 1.2 medium followed by 8 hours in pH 6.8 medium, 900 mL, 37° C., 50 rpm, n=6. It can also be seen that a zero order or near zero order release pattern is provided with 2.5% carbomer 934, while there is essentially no difference in the extended release profile when the amount of carbomer 934 is increased from 5 to 10%. In fact, unexpectedly, the higher amount of carbomer 934 caused the drug to be released more rapidly than that achieved with the 2.5% carbomer 934 extrudate.

### Examples 14-17

In these examples polymer, plasticizer, API and a release rate modifier were included.

### Example 14

| Component | Percent | |
|---|---|---|
| Acryl-EZE | 56% | |
| TEC | 14% | (25% based on |
| | | Acryl-EZE) |
| Theophylline | 20% | |
| Methocel K4M | 10% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.36 | Amp |
| Pressure | 2100 | PSI |

Comments: no flow in screw

### Example 15

| Component | Percent | |
|---|---|---|
| Acryl-EZE | 60% | |
| TEC | 15% | (25% based on |
| | | Acryl-EZE) |
| Theophylline | 20% | |
| Methocel K4M | 5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.32 | Amp |
| Pressure | 1700 | PSI |

Comments: flow problems in screw, but good product

### Example 16

| Component | Percent | |
|---|---|---|
| Acryl-EZE | 62% | |
| TEC | 15.5% | (25% based on |
| | | Acryl-EZE) |
| Theophylline | 20% | |
| Methocel K4M | 2.5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.3 | Amp |
| Pressure | 1500 | PSI |

Comments: good flow, flexible product

### Example 17

| Component | Percent | |
|---|---|---|
| Acryl-EZE | 64% | |
| TEC | 16% | (25% based on |
| | | Acryl-EZE) |
| Theophylline | 20% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.28 | Amp |
| Pressure | 1200 | PSI |

Comments: excellent flow, flexible product

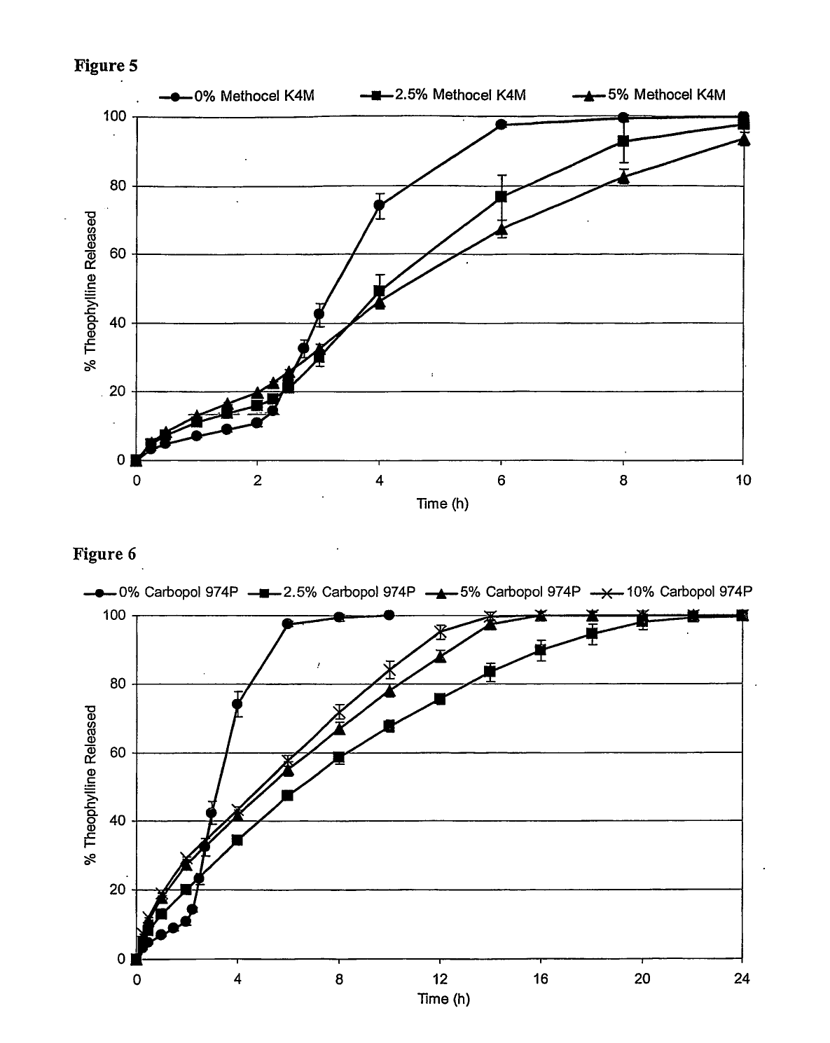

Referring now to FIG. 5, the influence of Methocel K4M on the theophylline release profile of thermoformed tablets prepared as described above is illustrated. The baseline formulation is the product of Example 17, which is the same formulation as Example 7 but with different processing parameters. The dissolution conditions: 2 hours in 0.1 N HCl medium followed by 8 hours in pH 6.8 medium, 900 mL, 37° C., 50 rpm, n=6.

It can be seen that the higher screw speed, drive amps and pressure employed in Example 17 decreased the amount of theophylline released between 0 and 2 hours of dissolution testing as compared to the product of Example 7. The dissolution profile of the product of Example 7 is illustrated in FIGS. 2-4. It can also be seen that Methocel decreased the rate of drug release between 2 and 10 when compared to the baseline formulation. Furthermore, the tablets containing 5% Methocel exhibited a more significantly reduced drug release rate in the pH 6.8 medium when compared to the product containing 2.5% Methocel. However, increasing the concentration of Methocel increased drug release during the 0.1 N HCl medium.

### Examples 18-20

In these examples polymer, plasticizer, API and a release rate modifier were included. (Continued on next page)

### Example 18

| Component | Percent | |
|---|---|---|
| Acryl-EZE | 56% | |
| TEC | 14% | (25% based on |
| | | Acryl-EZE) |
| Theophylline | 20% | |
| Carbopol 974P | 10% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.33 | Amp |
| Pressure | 1600 | PSI |

Comments: some reduced flow, flexible product, rough surface

### Example 19

| Component | Percent | |
|---|---|---|
| Acryl-EZE | 60% | |
| TEC | 15% | (25% based on |
| | | Acryl-EZE) |
| Theophylline | 20% | |
| Carbopol 974P | 5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.33 | Amp |
| Pressure | 1500 | PSI |

Comments: good flow, flexible product

### Example 20

| Component | Percent | |
|---|---|---|
| Acryl-EZE | 62% | |
| TEC | 15.5% | (25% based on |
| | | Acryl-EZE) |
| Theophylline | 20% | |
| Carbopol 974P | 2.5% | |

Processing Parameters

| | | |
|---|---|---|
| Zone 1 | 90° | C. |
| Zone 2 | 105° | C. |
| Zone 3 | 110° | C. |
| Die | 115° | C. |
| Screw Speed | 20 | rpm |
| Drive Amps | 0.3 | Amp |
| Pressure | 1400 | PSI |

Comments: excellent flow, flexible product

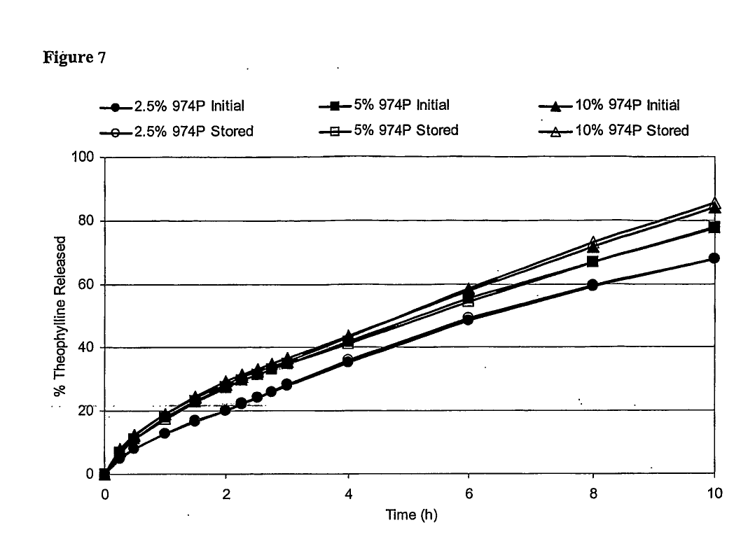

Referring now to FIG. 6, the influence of Carbopol 974P on the theophylline release rate from melt-extruded tablets prepared as described in Examples 18-20 is illustrated. The dissolution conditions: 2 hours in 0.1 N HCl followed by 22 hours in pH 6.8 medium, 900 mL, 37° C., 50 rpm, n=6. The baseline formulation was again the product of example 17, e.g. 0% Carbopol 974P, 20% Theophylline, 64% Acryl-EZE, 16% TEC (25% based on Acryl-EZE).

As noted with the extrudates containing carbomer 934, the presence of Carbopol 974P significantly extended the time over which theophylline was released. Tablets containing 2.5% Carbopol sustained drug release for approximately 20 hours of dissolution testing. The dissolution profiles of products containing 5 or 10% Carbopol were not significantly different, exhibiting near zero order release patterns and attaining complete drug release after approximately 14 hours of testing. In all examples, the presence of Carbopol increased the amount of theophylline released in the 0.1 N HCl medium.

Turning now to FIG. 7, the stability of theophylline release rate from melt-extruded Acryl-EZE matrix tablets containing Carbopol 974P is illustrated upon storage for 3 months at 40° C./75% RH in induction sealed HDPE containers with silica desiccant. The formulations studied were the products of Examples 14, 15 and 16. The dissolution conditions: 2 hours in 0.1 N HCl medium followed by 8 hours in pH 6.8 medium, 900 mL, 37° C., 50 rpm, n=6. It can be seen that tablets containing 2.5, 5 and 10% Carbopol were stable upon storage at accelerated conditions as the initial and stored dissolution profiles were superimposable.

### Example 21

The data generated from running the content uniformity tests described in paragraph [0058] for compositions prepared in the above in Examples are set forth below:

| | Percent of | |
|---|---|---|
| | Theoretical | |
| | Drug Content | Standard |
| Formulation | (n = 6) | Deviation |
| 20% Guaifenesin, 60° C., 80° C., 80° C., | 98.1 | 1.2 |
| 70° C., Ex. 5 | | |
| 20% Guaifenesin, 60° C., 85° C., 90° C., | 100.8 | 1.6 |
| 70° C., Ex. 6 | | |
| 20% Theophylline, 25% TEC (based on | 104.3 | 1.1 |
| Acryl-EZE), Ex. 7 | | |
| 30% Theophylline, 25% TEC (based on | 100.0 | 0.9 |
| Acryl-EZE) Ex. 8 | | |
| 5% GMS 191, 20% Theophylline, 25% | 104.1 | 0.6 |
| TEC (based on Acryl-EZE) Ex. 10 | | |
| 10% GMS 191, 20% Theophylline, 25% | 101.6 | 1.4 |
| TEC (based on Acryl-EZE) Ex. 9 | | |
| 2.5% Carbomer 934, 20% Theophylline, | 102.0 | 0.4 |
| 25% TEC (based on Acryl-EZE) Ex. 13 | | |
| 5% Carbomer 934, 20% Theophylline, | 102.8 | 0.1 |
| 25% TEC (based on Acryl-EZE) Ex. 12 | | |
| 10% Carbomer 934, 20% Theophylline, | 98.8 | 1.5 |
| 25% TEC (based on Acryl-EZE) Ex. 11 | | |
| 2.5% Methocel K4M, 20% Theophylline, | 102.7 | 0.8 |
| 25% TEC (based on Acryl-EZE) Ex. 16 | | |
| 5% Methocel K4M, 20% Theophylline, | 101.3 | 1.0 |
| 25% TEC (based on Acryl-EZE) Ex. 15 | | |
| 2.5% Carbopol 974, 20% Theophylline, | 101.6 | 1.0 |
| 25% TEC (based on Acryl-EZE) Ex. 20 | | |
| 5% Carbopol 974, 20% Theophylline, 25% | 102.4 | 1.1 |
| TEC (based on Acryl-EZE) Ex. 19 | | |
| 10% Carbopol 974, 20% Theophylline, | 101.3 | 1.0 |
| 25% TEC (based on Acryl-EZE) Ex. 18 | | |

The above data shows that the methods of the present invention provide products which are reproducible and highly consistent in drug content uniformity. The process is highly efficient, even when liquid TEC and carbomer were included. The products can be made in a single pass through the extruder.

While there have been described what are presently believed to be the preferred embodiments of the invention, those skilled in the art will realize that changes and modifications may be made thereto without departing from the spirit of the invention. It is intended to claim all such changes and modifications that fall within the true scope of the invention.

We claim:

1. A thermoformable composition, comprising:
i) a thermoformable acrylic polymer binder containing an acrylic resin, comprising
a) from 20 to 85 percent by weight of at least one alkyl acrylate or alkyl methacrylate moiety;
b) from 80 to 15 percent by weight of at least one vinyl or vinylidene moiety having a carboxylic acid group capable of salt formation; and
c) from 0 to 30 percent by weight of at least one other vinyl or vinylidene moiety copolymerizable with a) and b); and
ii) an effective amount an acrylic polymer plasticizer.

2. The composition of claim 1, further comprising a pharmaceutically active composition.

3. The composition of claim 1, farther comprising an alkalizing agent capable of reacting with said acrylic resin such that, after reaction, 0.1 to 10 mole percent of the acidic groups in i) b) are present in the salt form.

4. The composition of claim 1, further comprising a detackifier.

5. The composition of claim 1, wherein said plasticizer is present in an amount of from about 4.0 to about 40% by weight.

6. The composition of claim 5, wherein said plasticizer is present in an amount of from about 7 to about 35% by weight.

7. The composition of claim 6, wherein said plasticizer is present in an amount of from about 10 to about 30% by weight.

8. The composition of claim 1, wherein said plasticizer is selected from the group consisting of triethylcitrate, glyceryltriacetate, glyceryl monostearate, acetyltriethylcitrate, dibutyl sebacate, diethylphthalate, polyethylene glycol, glycerol, castor oil, and mixtures thereof.

9. The composition of claim 1, wherein said plasticizer is triethylcitrate.

10. The composition of claim 1, wherein said extrudable acrylic polymer binder is present in an amount ranging from about 10 to about 80% by weight.

11. The composition of claim 1, wherein said extrudable acrylic polymer binder is present in an amount ranging from about 15 to about 70% by weight.

12. The composition of claim 1, wherein said extrudable acrylic polymer binder is present in an amount ranging from about 20 to about 60% by weight.

13. The composition of claim 1, wherein the amount of the pharmaceutically active composition is from about 0.001 to about 85% by wt.

14. The composition of claim 1, wherein the amount of the pharmaceutically active composition is from about 1.0 to about 60% by wt.

15. The composition of claim 4, wherein the detackifier is selected from the group consisting of talc, aluminum hydrate, glyceryl monostearate, kaolin, and mixtures thereof.

16. The composition of claim 1, further including a member of the group consisting of pigments, flow aids, surfactants, anti-agglomerating agents, secondary binders, secondary detackifiers and mixtures thereof.

17. The composition of claim 1, further comprising a release rate modifier.

18. The composition of claim 17, wherein said release rate modifier is selected from the group consisting of hydroxypropylcellulose (HPC), poly(ethylene oxide) (PEO), hydroxypropyl methylcellulose (HPMC), ethylcellulose, cellulosic polymers, acrylic polymers, fat, waxes, lipids, and mixtures thereof.

19. The composition of claim 17, wherein said release rate modifier is selected from the group consisting of polycarbophils, carbomers, polysaccharides and mixtures thereof.

20. The composition of claim 17, wherein said controlled release rate modifier an amount ranging from about 1 to about 40% by weight.

21. The composition of claim 20, wherein said controlled release rate modifier an amount ranging from about 2 to about 30 weight %.

22. The composition of claim 16, wherein said pigment is selected from the group consisting of FD&C and D&C lakes, titanium dioxide, magnesium carbonate, talc, pyrogenic silica, iron oxides, channel black, riboflavin, carmine 40, curcumin, annatto, insoluble dyes, pearlescent pigments based on mica and/or titanium dioxide and mixtures thereof.

23. The composition of claim 16, wherein said flow aid is selected from the group consisting of silica, alumina, silicon dioxide. talc, stearic acid, and metallic stearates and the surfactant or wetting agent is selected from the group consisting of sodium lauryl sulfate, dioctyl sodium sulfosuccinate, polysorbates, Tween 80, copolymers of propylene oxide, ethylene oxide, sucrose stearate, cremophor, emulphor, glycerine, PEG, PPG and hydrophilic surfactants having an HLB of >10 and mixtures thereof.

24. The composition of claim 1, wherein alkyl acrylate is ethyl acrylate and the vinyl or vinylidene moiety having a carboxylic acid group capable of salt formation is methacrylic acid. and the alkalizing agent is selected from the group consisting of bicarbonates, carbonates, phosphates, and hydroxides of sodium or potassium, magnesium carbonate, magnesium hydroxide, ammonium carbonate, ammonium bicarbonate, magnesium oxide, calcium hydroxide, or mixtures thereof.

25. The composition of claim 16, wherein said anti-agglomeration agent is kaolin.

26. The composition of claim 16, wherein the secondary binder is selected from the group consisting of xanthan gum, sodium alginate, propylene glycol alginate, hydroxypropylmethylcellulose (HPMC), hydroxyethylcellulose (HEC), sodium carboxymethylcellulose (sodium CMC), polyvinylpyrrolidone (PVP), Konjac flour, carrageenan, pregelatinized starch, other film-forming polymer and mixtures thereof.

27. The composition of claim 16, wherein the secondary detackifier is selected from the group consisting of sodium sulfate, calcium sulfate, calcium chloride, other inorganic or organic water-sequestering agents and mixtures thereof.

28. A method of preparing a thermoformed composition comprising:
i) combining a thermoformable acrylic polymer binder containing an acrylic resin, comprising:
a) from 20 to 85 percent by weight of at least one alkyl acrylate or alkyl methacrylate moiety;
b) from 80 to 15 percent by weight of at least one vinyl or vinylidene moiety having a carboxylic acid group capable of salt formation; and
c) from 0 to 30 percent by weight of at least one other vinyl or vinylidene moiety copolymerizable with a) and b);
with an effective amount of an acrylic polymer plasticizer; and
ii) extruding the mixture obtained as result of step i) in an extruder.

29. The method of claim 28, further comprising admixing a pharmaceutically active composition with said extrudable acrylic polymer and said plasticizer prior to said extruding of said mixture.

30. The method of claim 28, further comprising admixing an alkalizing agent capable of reacting with said acrylic resin such that, after reaction, 0.1 to 10 mole percent of the acidic groups in i) b) are present in the salt form with said extrudable acrylic polymer and said plasticizer prior to said extruding of said mixture.

31. The method of claim 28, further comprising admixing a detackifier with said extrudable acrylic polymer and said plasticizer prior to said extruding of said mixture.

32. The method of claim 28, further comprising admixing a member of the group consisting of release rate modifiers, pigments, flow aids, surfactants, anti-agglomerating agents, secondary binders, secondary detackifiers and mixtures thereof with said extrudable acrylic polymer and said plasticizer prior to said extruding of said mixture.

33. The method of claim 28, wherein said extruding is carried out in a single screw extruder having a feed zone, a compression zone, a mixing zone and an exit die.

34. The method of claims 28-33, further comprising spheronizing the resultant extruded mixture exiting the extruder.

35. The method of claims 28-33, further comprising pelletizing the extruded mixture exiting the extruder.

36. The method of claims 28-35, further comprising milling the resultant extruded mixture exiting the extruder.

37. The method of claims 28-35, further comprising cooling the extruded mixture exiting the extruder.

38. The method of claims 28-37, further comprising compressing the resultant extruded mixture into a pharmaceutical dosage form.

39. The method of claim 28-37, further comprising encapsulating the resultant extruded mixture.

40. A pharmaceutical dosage form containing a product obtained by the method of any of claims 28-37.

41. A pharmaceutical dosage form comprising the composition of claim 2.

42. The pharmaceutical dosage form of claim 40 or 41 having zero order or near zero order release characteristics.

43. The pharmaceutical dosage form of claim 40 or 41 further comprising a film coating.

44. The pharmaceutical dosage form of claim 40 or 41 wherein said pharmaceutically active composition is released therefrom over an 8 to 24 hour period after administration to a patient in need of said pharmaceutically active composition.

45. A thermoformable composition comprising:
i) an extrudable acrylic polymer binder containing an acrylic resin, comprising
a) from 20 to 85 percent by weight of at least one alkyl acrylate or alkyl methacrylate moiety;
b) from 80 to 15 percent by weight of at least one vinyl or vinylidene moiety having a carboxylic acid group capable of salt formation; and
c) from 0 to 30 percent by weight of at least one other vinyl or vinylidene moiety copolymerizable with a) and b); and
ii) an effective amount of a plasticizer.

46. The composition of claim 45, further comprising a pharmaceutically active composition.

47. A thermoformable composition comprising:
a) from about 20 to about 80% by weight Eudragit L100-55;
b) from about 15 to about 60% by weight triethyl citrate; and
c) from about 19 to about 76% by weight talc.
